(19) United States
(12) Patent Application Publication
(10) Pub. No.: US 2012/0285471 A1
(43) Pub. Date: Nov. 15, 2012

(54) Holster for oxygen supply line tubes

(75) Inventor: Marc Eric Keesy, Eagle River, WI (US)

(73) Assignee: Marc Eril Keesy, Eagle River, WI (US)

(21) Appl. No.: 13/068,362

(22) Filed: May 10, 2011

(51) Int. Cl. A61M 16/00

(52) U.S. Cl. 128/207.18

(57) ABSTRACT

This invention is a device designed to eliminate the discomfort and irritation behind the ears caused from oxygen supply line tubes by having the oxygen supply line tube wrapped on the holster and off the ears and securely in place.

### CROSS-REFERENCE TO RELATED APPLICATIONS

Not Applicable

### STATEMENT REGARDING FEDERALLY SPONSORED RESEARCH OR DEVELOPMENT

Not Applicable

### REFERENCE TO SEQUENCE LISTING, A TABLE, OR A COMPUTER PREGRAM LISTING COMPACT DISC APPENDIX

Not Applicable

### BACKGROUND OF THE INVENTION

I made this invention for my Mother who was prescribed oxygen from her Doctor. An oxygen tank through a supply line tube that has a loop at its end administers the oxygen. In the center of the loop there are two-nostril ports. These ports are inserted into the nostril and the loop wraps over the ears and under the chin then adjusted with the adjustment knob.

My Mother's ears became very sore so I made this device. It keeps the oxygen supply line tube off the ears and staying securely in place I achieved this by making a tube holster and attaching a barrette to tube holster and put the barrette in her hair. This worked fine but when her hair started to thin I refined the idea to make the tube holster available for bald or people with thinning hair. I made a left side and a right side holster for the tubing with a strap (The strap can be fixed or adjustable) between them. The strap crosses over the head and the holsters set above and behind the ears or (This is the way we use it mostly) strap can be placed around the back of the neck and the tube holster behind and under the ears. The oxygen supply line tube is used in the standard way except for the loop wrapping over the holster and off the ears.

Then I showed the invention to my brother whom is a Registered Nurse at a Nursing Home and told me that there is nothing out there for people on oxygen and the sores behind the ears is a very serious problem at his workplace. The sore behind the ears have been address by placing cotton between the ears and tubing but did not relieve the pressure on the ear.

### BRIEF SUMMARY OF THE INVENTION

This invention is a device designed to eliminate the irritation behind the ears caused from oxygen supply line tubes by having the oxygen supply line tube wrapped on the holster and off the ears and securely in place. I achieved this by making a tube holster and attaching a barrette to tube holster and put the barrette in their hair, for bald or people with thinning hair the holsters with a strap (The strap can be fixed or adjustable) between them.

The strap crosses over the head and the holsters set above and behind the ears or strap can be placed around the back of the neck and the tube holster behind and under the ears.

### BRIEF DESCRIPTION OF THE SEVERAL VIEWS OF THE DRAWING

FIG. 1, The holster for oxygen supply line tube, (a) adjustable strap with a fastener, (b) slotted area of the holster to receive strap, (c) the channeled holster for oxygen supply line tube, (d) the oxygen supply line tube.

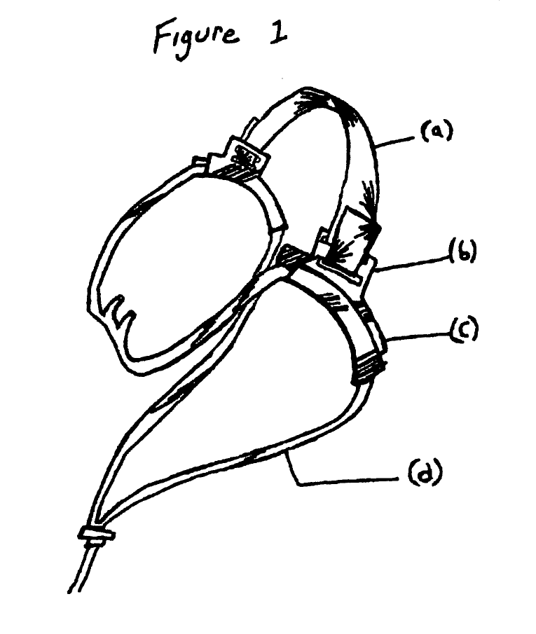

### DETAILED DESCRIPTION OF THE INVENTION

The holster for oxygen supply line tube basic function is to keep the oxygen supply line tube off the ears. The holster for oxygen supply line tube has two basic parts. 1.

The holster. 2. The strap or barrettes to keep the holster securely in place. The design style can have many variations but the function is this, a channeled area for the oxygen supply line tube and a means to hold the channeled area in place. For this detailed description of the invention I describe the way I made my prototype.

The holster can be made of various materials such as types of plastics, rubber, etc. This material must keep its form either rigid or flexible. There are many ways to manufacture such as injection molding, pour molded, hand form molded, etc. The strap can be made from many materials such as various fabrics, skins, elastic band, rigid material (plastic, metal), and etc. The strap can be adjustable with fasteners at the ends for example Velcro or can be fixed to the holster at a standard size with an elastic band or rigid material. There are many types of barrette and they can be fastened to a short strap by Velcro or other means and then fastened to the holster by the same method as the strap.

For my prototype I made it as follows:

I cut out a paper patterns; the front and back patterns at the bottom length is 2.5″ up the curve. (I used a 6″ diameter circle to achieve the proper arch) The front pattern is a 0.375″wide arch. The back pattern is 0.625″wide arch with an area strap attachment. From the top right side measure down the arch 0.75″ starts the area for the strap attachment and the top left side measure up the arch 1.0625″ ends the area for the strap attachment. 1.125″ sides with the top measurement of 1.375″. Cut the pattern from the 0.75″ measurement and the 1.0625″ from 0.625″width to an angle 0.375″wide at each end. To cut the slotted area for strap attachment. The slotted areas are, starting at the 1 0.375″ flat top measure down 0.25″′ and 0.125″ from right side cut a 1″ by .125″ slot. At the bottom of the slot measure down 0.75″ and cut another 1″ by 0.125″ slot parallel with the upper slot.

The final pattern is the bottom arch that will attach to the front and back pieces. The final pattern is 2.5″ long by 0.3125″.

Take some Epoxy putty and mixes it according to the instruction then roll it flat to 0.0625″ on wax paper the place the three pattern piece on the rolled out mixed putty and cut out with a razor knife remove from the wax paper and remove the pattern pieces then take the bottom piece and affix it to the back side edge of bottom arch by applying pressure and fusing the two edges. Then attach the front side bottom arch edge to the remaining edge of the bottom piece edge by applying pressure and fusing the two edges. Make sure to keep the channel open while the putty mixture cures. You can use a 0.25″ spacer. Sand smooth and round bottom and edges. Now you have made one side of the holster the make the other side simply flip the pattern sides over and repeat the manufacturing process.

To attach the strap to the holsters I use a 1″ wide by 10.5″ piece of knitted fabric and threaded 3″ of the end over the top (1.375″ top flat) into the top slot out the back side of the holster and into the bottom slot and over the top of the threaded fabric and with a needle and thread stitched the two together securely. Next I repeated the process to the other side of the fabric and holster. I did not make this one adjustable because I used my mother as the model.

To use the Holster for oxygen supply line tube (note that the back side 0.75″ distance from the top edge to the strap area should be top or up) place the strap around the back of the neck and wrap the oxygen supply loop into the channels of the holster and the nostril ports into the nostril and adjust the oxygen supply line with adjustment knob on the oxygen supply line under the chin to fit.

You can follow the instruction that are supplied with the oxygen supply line tube with the exception of wrapping the looped area of the oxygen supply line tube over the ears and amend it to wrapping the looped area of the oxygen supply line tube over the oxygen supply line tube holster.

1. What I claim my invention is to removes the use of wearing an oxygen supply line tube from around the ears relieving the discomfort and irritation by the way of my invention the holster for oxygen supply line tube to keep the oxygen supply line tube off the ears and nostril ports in nostril and securely in place: The holster for oxygen supply line tube design style can have many variations but the function is this, a channeled area (holster) for the oxygen supply line tube and a means to hold the channeled area (holster) in place.
